(19) United States
(12) Patent Application Publication
(10) Pub. No.: US 2025/0325405 A1
(43) Pub. Date: Oct. 23, 2025

(54) THUMBTACK NEEDLE DRESSING FOR HEMOSTASIS AFTER OPERATION

(71) Applicant: Shanghai Sixth People's Hospital, Shanghai (CN)

(72) Inventors: Cunyi Fan, Shanghai (CN); Binbin Xu, Shanghai (CN); Xu Wang, Shanghai (CN); Ai Yang, Shanghai (CN); Chunxin Li, Shanghai (CN); Chao Zhou, Shanghai (CN); Bing Tu, Shanghai (CN); Yuxin Gao, Shanghai (CN); Wen Yang, Shanghai (CN)

(21) Appl. No.: 18/950,187

(22) Filed: Nov. 18, 2024

Related U.S. Application Data
(63) Continuation of application No. PCT/CN2024/118940, filed on Sep. 14, 2024.

(30) Foreign Application Priority Data
Apr. 19, 2024 (CN) 202410476246.X

(51) Int. Cl. A61F 13/00; A61B 17/12

(52) U.S. Cl. CPC A61F 13/00063; A61B 2017/12004

(57) ABSTRACT

A thumbtack needle dressing for hemostasis after an operation includes a dressing body and a hollow thumbtack needle. The dressing body includes a patch layer, a drug groove and a hemostatic drug layer connected in sequence. The hollow thumbtack needle includes a needle body and a needle tip, the needle body is communicated with the drug groove, the hollow thumbtack needle penetrates through the hemostatic drug layer to make the needle body and the needle tip expose from the hemostatic drug layer, and a needle cap is further provided on the needle tip. The drug groove is provided with a drug, and the hemostatic drug layer is a self-adhesive layer provided with a hemostatic drug. The patch layer is provided with a fixing strap to adjust and fit. The thumbtack needle dressing can achieve precise drug delivery and localized high-concentration medication.

### TECHNICAL FIELD

The disclosure relates to the technical field of medical supplies, and more particularly to a thumbtack needle dressing for hemostasis after an operation.

### BACKGROUND

In the medical field, the treatment of a wound bleeding after an operation is crucial. Hemostatic dressings of the related art are usually made of gauze, cotton balls, or other absorbent materials, which achieve hemostasis by applying pressure to the wound or absorbing blood.

For example, a hemostatic patch is equipped with a hemostatic cotton, a pressure adjustment device, and a pull ring on the hemostatic patch body. When hemostasis is performed on a bleeding point of a patient, the hemostatic patch body can adhere to the skin.

However, the hemostatic dressings of the related art have the following drawbacks.

(1) Limited hemostatic effect: the hemostatic dressings of the related art have certain limitations in terms of hemostatic speed and hemostatic effect, and cannot meet the needs of the patient for rapid hemostasis after the operation.

(2) Easy to fall off or shift: the hemostatic dressings of the related art are easy to fall off or shift after the operation, which leads to reduced or ineffective hemostasis. Frequent replacement and re-fixation of the hemostatic dressings are required, thereby increasing additional waste and corresponding costs.

(3) Patient comfort is not high: dressings and hemostatic materials of the related art cause discomfort to the patient, such as a feeling of tightness and stuffiness, which affects the comfort and recovery of the patient after the operation.

(4) The wound is difficult to heal: the hemostatic dressings of the related art adhered to the wound create a stuffy and airtight environment in the wound, and exudation of tissue fluid at the wound can breed bacteria and cause infection.

(5) Dressing patch is not easy to replace: the hemostatic dressings of the related art can tear the wound when they are removed and replaced, which causes secondary damage, increases patient discomfort, is not conducive to the wound healing and has a negative impact on doctor-patient relationship.

Thumbtack needle is a general name for the thumbtack needle type intradermal needle, which resembles a thumbtack. According to the skin theory of traditional Chinese medicine, a thumbtack needle applied to acupoints of limbs and abdomen can stimulate meridians and treat diseases. Modern medicine has also confirmed through experiments that thumbtack needle can continuously stimulate nerve endings through a weak and lasting therapeutic effect, thereby enhancing the function of viscera and continuously improving the functions of nervous system, digestive system, endocrine system, and so on. The advantage of the thumbtack needle therapy lies in a short and small size of the needle body, which does not penetrate deeply into the subcutaneous tissue, making it less likely to damage important nerves and blood vessels. The level of pain experienced by the patient is also lower than that of general acupuncture therapy, with minimal bleeding. There are almost no adverse events such as fainting from needles, needle breakage, or needle bending, making it more readily accepted by the patient. The thumbtack needle therapy has a long needle retention time, featuring the characteristic of “leaving the needle to await the arrival of Qi”. Prolonged stimulation of acupoints can have a dual-regulation effect.

Therefore, combining the thumbtack needle with the hemostatic dressings to design a thumbtack hemostatic dressing will help achieve rapid, stable, and highly effective hemostasis.

### SUMMARY

The purpose of the disclosure is to provide a thumbtack needle dressing for hemostasis after an operation during hospitalization, aiming at the defects existing in hemostatic dressings of the related art, and the thumbtack needle dressing combines a thumbtack needle with hemostatic materials to achieve the goal of rapid, stable and efficient hemostasis.

In order to achieve the aforementioned purpose, the disclosure provides the thumbtack needle dressing for hemostasis after the operation, which includes a dressing body and a hollow thumbtack needle. The dressing body includes a patch layer, a drug groove and a hemostatic drug layer sequentially connected in that order;
the hollow thumbtack needle includes a needle body and a needle tip, the needle body is communicated with the drug groove, the hollow thumbtack needle penetrates through the hemostatic drug layer to make the needle body and the needle tip expose from the hemostatic drug layer, and a needle cap is further provided on the needle tip. The drug groove is provided with a drug therein, and the hemostatic drug layer is a self-adhesive layer provided with a hemostatic drug; and the patch layer is provided with a fixing strap to adjust and fit.

In an embodiment, the hollow thumbtack needle is vertically connected to the drug groove, and the needle body is communicated with a middle part of the drug groove.

In an embodiment, the hemostatic drug in the hemostatic drug layer includes at least one selected from the group consisting of tranexamic acid, aminocaproic acid, and etamsylate.

In an embodiment, the hemostatic drug layer is made of a material added with at least one selected from the group consisting of dextrin, starch slurry, and hydroxypropyl cellulose.

In an embodiment, the number of fixing straps is two, and the two fixing straps provided on two sides of the patch layer are adhesive tapes or bandages.

In an embodiment, the patch layer is made of a medical adhesive tape, non-woven fabric, or a medical silicone material.

In an embodiment, the drug in the drug groove is an antimicrobial and/or anti-inflammatory drug.

In an embodiment, the drug groove is made of one selected from the group consisting of polymer materials (such as polyvinyl chloride (PVC), etc.), stainless steel, and aluminum.

Compared to the related art, the benefits of the disclosure are as follows.

(1) Rapid hemostasis: through a design of the hemostatic drug in the drug layer, physical compression hemostasis is combined with drug action hemostasis to make the thumbtack needle dressing have excellent hemostatic effects, and achieve the purpose of rapid hemostasis.

(2) Strong stability: through using the external fixing strap and a structural design where the thumbtack needle is connected with the patch drug groove, the dressing is effectively prevented from falling off or shifting during activities after the operation; this not only ensures durability of the hemostatic effect but also significantly reduces the risk of the wound bleeding again due to the movement of the dressing, thereby markedly enhancing the treatment effect of wound recovery after the operation.

(3) Improve patient comfort: after applying the dressing, the thumbtack needle can massage acupoints, which enhances the effect of treating common chronic pain diseases in orthopedics for the patient.

(4) Reduce bacterial infection in the wound: the design that the hollow thumbtack needle is connected to the drug groove allows the antimicrobial drug and the like to smoothly enter the patient's body; this reduces the risk of bacterial infection in the wound and helps promote wound healing.

(5) Improving treatment outcomes and patient compliance: Compared to the hemostatic dressings of the related art, the thumbtack needle dressing provided by the disclosure is easier to remove without causing secondary injury to the patient when removing the dressing.

### BRIEF DESCRIPTION OF DRAWINGS

FIG. 1 illustrates a schematic structural diagram of a thumbtack needle dressing for hemostasis after an operation provided by the disclosure.

FIG. 2 illustrates a schematic structural diagram of a sealing state of the thumbtack needle dressing for hemostasis after the operation provided by the disclosure.

FIG. 3 illustrates a schematic diagram of a needle cap.

### DESCRIPTION OF REFERENCE NUMERALS

1, dressing body; 2, hollow thumbtack needle; 3, patch layer; 4, drug groove; 5, hemostatic drug layer; 6, fixing strap; 21, needle body; 22, needle tip; 23, needle cap.

### DETAILED DESCRIPTION OF EMBODIMENTS

In order to make the disclosure clearer and more understandable, preferred embodiments are described in detail below, in conjunction with the accompanying drawing.

### Embodiment 1

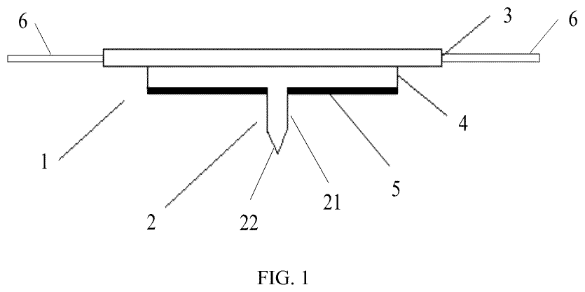

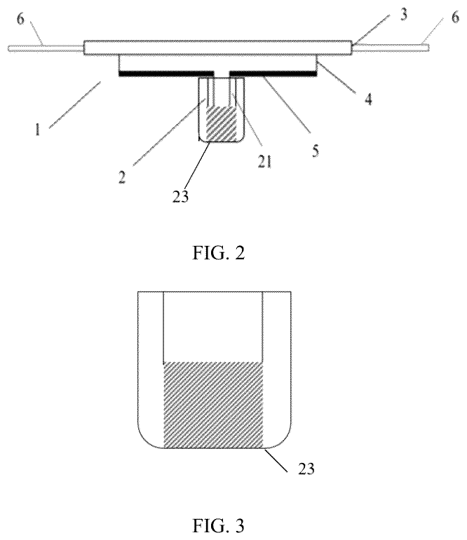

A thumbtack needle dressing for hemostasis after an operation, as shown in FIGS. 1-3, includes a dressing body 1 and a hollow thumbtack needle 2. The dressing body 1 includes a patch layer 3, a drug groove 4 and a hemostatic drug layer 5 sequentially connected in that order. The patch layer 3 completely covers the end surface of drug groove 4 (the end surface where patch layer 3 connects the drug groove 4, shown as a top surface of the drug groove in FIG. 1) to form a seal for the drug groove, and the hemostatic drug layer 5 covers the other end surface of the drug groove (shown as a bottom surface of the drug groove in FIG. 1). The hollow thumbtack needle 2 includes a needle body 21 and a needle tip 22, and the hollow thumbtack needle 2 is communicated with the middle part of the drug groove 4 and is perpendicular to the drug groove 4. The hollow thumbtack needle 2 penetrates through the hemostatic drug layer 5 to make the needle body 21 and the needle tip 22 expose from the hemostatic drug layer, and a needle cap 23 made of silicone is also provided on the needle tip 22. The drug groove 4 is equipped with an antimicrobial drug, the hemostatic drug layer 5 is a self-adhesive layer provided with a hemostatic drug, and the hemostatic drug is selected as tranexamic acid. The hemostatic drug layer is in direct contact with the skin of a patient's wound, which effectively inhibits the bleeding condition at the wound and achieves a faster hemostasis effect. The patch layer 3 is made of a medical adhesive tape or non-woven fabric, and it also is made from a soft and breathable medical-grade silicone material to form a fixed patch layer. The patch layer has sufficient elasticity and adaptability to closely adhere to the skin of the patient. The number of the fixing straps is two, and the two fixing straps 6 provided on two sides of the patch layer 3 are adhesive tapes. Since the drug groove 4 is communicated with the thumbtack needle 2, the antimicrobial drug contained within the drug groove 4 is released through the hollow thumbtack needle, and the antimicrobial drug is pre-injected into the drug groove and stored within it. The needle tip 22 is sealed with the needle cap 23, which is removed when in use. The antimicrobial drug is delivered to the wound through a hollow part of the needle, thereby reducing the risk of bacterial infection in the wound of the patient.

Adhesiveness of the hemostatic drug layer is variable, and a material of the hemostatic drug layer is a hydrogel. The material of the hemostatic drug layer is added with at least one selected from the group consisting of dextrin, starch slurry, and hydroxypropyl cellulose. As the usage time increases, the adhesiveness gradually decreases, which facilitates the replacement or removal of the thumbtack needle dressing.

In the embodiment, preferably, the hemostatic drug layer is a hydrogel layer, and the hydrogel layer contains the hemostatic drug tranexamic acid and the excipient hydroxypropyl cellulose. The hemostatic layer production scheme is as follows:
Step 1: Dissolve the photoinitiator in water, stir thoroughly to form solution A;
Step 2: Dissolve the methylacrylylated gelatin in solution A and stir thoroughly to form mixed solution B;
Step 3: Dissolve hydroxypropyl cellulose in mixed solution B and stir thoroughly to form mixed solution C;
Step 4: Dissolve tranexamic acid in mixed solution C and stir thoroughly to form mixed solution D;
Step 5: Pour the mixed solution D into the hemostatic drug layer mold to obtain a liquid hemostatic drug layer;
Step 6: Irradiate the liquid hemostatic drug layer with ultraviolet light with a wavelength of 365 nm for 5-10 minutes, and obtain the hemostatic drug layer after the photocrosslinking is sufficient.

In the above process, the ratio of the amount of each raw material added is: the mass ratio of photoinitiator, methylacrylylated gelatin, hydroxypropyl cellulose and tranexamic acid in the solvent is 1:2:1:2.

The drug groove is made of one selected from the group consisting of polymer materials (such as PVC, etc.), stainless steel, and aluminum.

The thickness of patch layer 3 is 0.2 millimeter (mm) to 2 mm, the height of the drug groove is 0.5 mm to 2 mm, and the thickness of the drug layer is 0.1 mm to 0.5 mm. The length of the hollow thumbtack needle is 2 mm to 3 mm, and the insertion depth of the hollow thumbtack needle into the body is 0.15 mm to 0.25 mm. Specific dimensions can vary depending on an actual usage scenario.

The duration of use for the thumbtack needle dressing is 48 to 72 hours, and it can be appropriately adjusted according to the actual situation of a user and the absorption rate of the drug in the drug patch layer.

Mode of use: the thumbtack needle dressing of the disclosure is applied onto the wound of the patient, and the adhesive tapes are adjusted to ensure that the thumbtack needle dressing is securely attached to the wound. The tiny needle of the thumbtack needle can be inserted into skin acupoints or the wound, which not only alleviates pain and other discomforts but also delivers a drug to the wound through the drug groove, achieving rapid hemostasis and promoting wound healing.

The thumbtack needle dressing provided by the disclosure is used for hemostasis, antibacterial treatment, and pain relief after the operation, providing strong support for the recovery of the patient after the operation.

### Embodiment 2

The thumbtack needle dressing for hemostasis after the operation, as shown in FIGS. 1-3, includes a dressing body 1 and a hollow thumbtack needle 2, the basic structure of the thumbtack needle dressing is the same as that of the embodiment 1. Different from embodiment 1, embodiment 2 uses aminocaproic acid as the hemostatic drug, and the drug in the drug groove is an anti-inflammatory drug. The two fixing straps 6 on two sides of the patch layer 3 are bandages. In the disclosure, through a hollow structure of the thumbtack needle, the anti-inflammatory drug can be injected according to actual needs and delivered to the wound through the hollow part of the needle when necessary. By adjusting the bandages, the dressing and the thumbtack structure can be ensured to closely adhere to the wound, preventing detachment or displacement.

Use effect: with the thumbtack dressing of the disclosure, precise drug delivery and localized high-concentration medication can be achieved. This not only helps with rapid hemostasis but also effectively reduces the risk of infection and promotes wound healing.

The disclosure provides an efficient, safe, and comfortable hemostatic dressing for use after the operation. By delivering precise medication and localized high-concentration drug administration, it promotes wound recovery and healing, offering strong support for the recovery of the patient after the operation.

### Embodiment 3

The thumbtack needle dressing for hemostasis after the operation, as shown in FIGS. 1-3, includes the dressing body 1 and the hollow thumbtack needle 2, the basic structure of the thumbtack needle dressing is the same as that of the embodiment 1. Different from embodiment 1, the hemostatic drug in this embodiment is etamsylate, the antimicrobial drug is arranged in the drug groove, and the two fixing straps 6 are provided on two sides of the patch layer 3 and are bandages.

The disclosure combines the hollow thumbtack needle with a carrier (equivalent to the hemostatic dressing), allowing the user to embed the needle at corresponding acupoints, which can act as an acupoint massage, alleviate pain at the wound site, promote the circulation of the organ's circulatory system, achieve a massage effect, and ultimately, the massage effect of the thumbtack needle can effectively relieve post-surgical pain and other discomforts of the patient.

The above embodiments are only preferred embodiments of the disclosure and are not intended to limit the disclosure in any formal or substantive way. It should be noted that those skilled in the art, without departing from the scope of the disclosure, can also make several improvements and supplements, which should also be considered within the scope of protection of the disclosure.

What is claimed is:

1. A thumbtack needle dressing for hemostasis after an operation comprising: a dressing body and a hollow thumbtack needle, wherein the dressing body comprises a patch layer, a drug groove and a hemostatic drug layer sequentially connected in that order;
wherein the hollow thumbtack needle comprises a needle body and a needle tip, the needle body is communicated with the drug groove, the hollow thumbtack needle penetrates through the hemostatic drug layer to make the needle body and the needle tip expose from the hemostatic drug layer, and a needle cap is further provided on the needle tip;
the drug groove is provided with a drug therein, and the hemostatic drug layer is a self-adhesive layer provided with a hemostatic drug; and
wherein the patch layer is provided with a fixing strap to adjust and fit.

2. The thumbtack needle dressing as claimed in claim 1, wherein the hollow thumbtack needle is vertically connected to the drug groove, and the needle body is communicated with a middle part of the drug groove.

3. The thumbtack needle dressing as claimed in claim 1, wherein the hemostatic drug in the hemostatic drug layer comprises at least one selected from the group consisting of tranexamic acid, aminocaproic acid, and etamsylate.

4. The thumbtack needle dressing as claimed in claim 1, wherein the hemostatic drug layer is made of a material added with at least one selected from the group consisting of dextrin, starch slurry, and hydroxypropyl cellulose.

5. The thumbtack needle dressing as claimed in claim 1, wherein the fixing strap is an adhesive tape or a bandage.

6. The thumbtack needle dressing as claimed in claim 1, wherein the patch layer is made of a medical adhesive tape, non-woven fabric, or a medical silicone material.

7. The thumbtack needle dressing as claimed in claim 1, wherein the drug in the drug groove is an antimicrobial and/or anti-inflammatory drug.

8. The thumbtack needle dressing as claimed in claim 7, wherein the drug groove is made of one selected from the group consisting of polymer materials, stainless steel, and aluminum.
